(19) United States
(12) Patent Application Publication
(10) Pub. No.: US 2010/0015243 A1
(43) Pub. Date: Jan. 21, 2010

(54) METHOD FOR PREPARING MATERIALS HAVING ANTITHROMBOTIC ACTIVITY FROM MUSKRAT MUSK AND MATERIALS OBTAINED FROM THE METHOD

(76) Inventors: Keun Ki Kim, Gyeongsangnam-do (KR); Han Seok Kang, Gyeongsangnam-do (KR); Yong Ju Choi, Chungcheongnam-do (KR); Teak Sooki Shin, Gyeongsangnam-do (KR); Seon Ku Kim, Daegu (KR); Jae Ho Lee, Gyeongsangnam-do (KR); Yong Gyun Kim, Gyeongsangnam-do (KR); Sang Yoon Jeon, Busan (KR)

Correspondence Address: DICKSTEIN SHAPIRO LLP, 1825 EYE STREET NW, Washington, DC, 20006-5403, US

(21) Appl. No.: 12/519,342

(22) PCT Filed: Dec. 17, 2007
(86) PCT No.: PCT/KR07/06583

§ 371 Date: Aug. 5, 2009

(30) Foreign Application Priority Data
Dec. 16, 2006 (KR) 10-2006-0129064

(51) Int. Cl. A61K 35/55

(52) U.S. Cl. 424/525

(57) ABSTRACT

Disclosed herein is a method of preparing an antithrombotic agent from muskrat musk and an antithrombotic agent having a high potency obtained therefrom, wherein the antithrombotic agent is prepared by treating muskrat musk with ethanol to obtain an ethanol extract; carrying out two normal phase column chromatographies while raising the combination ratio of hexane and ethyl acetate to obtain numerous fractions; carrying out thin layer chromatographies and identifying the material patterns of the fractions with UV lamps and 10% sulfuric acid to divide the materials which have a similar moving distance into groups; measuring a thrombin time of each group to isolate a group having a potent antithrombotic activity; carrying out a reverse phase column chromatography while raising the combination ratio of acetonitrile and water to obtain numerous fractions; and isolating an antithrombotic agent material having a high potency therefrom. According to the present invention which provides an antithrombotic agent having a high potency from muskrat musk, the value added of muskrat musk is raised.

### TECHNICAL FIELD

The present invention relates to a method of preparing an antithrombotic agent from muskrat musk and an antithrombotic agent having a high potency obtained therefrom. More specifically, the present invention relates to a method of preparing an antithrombotic agent from muskrat musk which comprises treating muskrat must with an organic solvent to obtain an organic solvent extract, carrying out two normal phase column chromatographies and one reverse phase column chromatography in order to obtain numerous fractions and measuring a thrombin time of each fraction, and an antithrombotic agent having a high potency obtained from the method.

### BACKGROUND ART

The muskrat (Ondatra zibethicus) is a mammalian which belongs to order Rodentia and family Muridae, is oval-shaped, has a body length of about 35 cm and a tail length of 25 cm and weighs about 1 kg. Muskrats are common near marsh and lake which is full of weed. They act from spring to late autumn and have a limited action in winter. They are herbivorous, but they also eat fish or aquatic animals. They practice monogamy and have a fine breeding. Female muskrats breed from the 4^{th} month to the 9^{th} month and have 2 or 3 litters a year. Male muskrats secrete musk through an accessory genital gland from two sachets located at the hypogastric, which is a symptom of sexual excitation for breeding. Muskrat musk is a skin-colored liquid and has a sweet-smelling and a male muskrat produces 5 to 8 g of muskrat musk per year on average.

Muskrat musk has a similar composition and pharmacological effect similar to that of the musk from a musk deer. Thus, it is worthy of notice as a medicinal raw material to replace the musk from musk deer.

Up to date, most of natural musk has been obtained from musk deer and used as a medicinal raw material. However, in a treaty of CITES (Convention on International Trade in Endangered Species), musk deer is classified into a threatened animal and designated as a first class animal for protection, and thus the slaughter and breeding of musk deer for harvesting musk is prohibited. Thus, civet obtained from civet cat is utilized as a medicinal raw material, but the obtainable amount is extremely small and, since civet was known as a source of SARS, it has been now slaughtered and disinfected.

Therefore, muskrat has a high potential to be developed as a new animal source of livestock industry. Further, since muskrat musk has a similar pharmacological effect to that of the musk from musk deer, it has a high potential to be developed as a medicinal raw material to replace the musk from musk deer and can be developed as cosmetics and perfumes.

Meanwhile, asprin which is generally known as having an antithrombotic effect prevents heart failure/blood coagulation, attenuates heart damage when heart attack strikes, and is effective in reducing pains such as muscle pains related to weight training, relieving pains of mild wounds, treating inflammation, alleviating fever, relieving headache, relieving chronic pains of knee and shoulder, etc. However, asprin has defects in that, since it can dilute blood, there is a risk of hemorrhagic stroke and, since it lowers digesting ability, when it is administered for a long time, a spasm of stomach can be attacked. Furthermore, when children and teenage youth are administered with asprin, they can suffer from a vital Reye's syndrome (a brain injury which is common in an infant).

Antithrombotic agent is administered for the prevention of cardiovascular diseases such as heart failure, stroke, etc., and there are market drugs such as Plavix tablet (Sanofi-Aventis), Asprinprotect tablet (Bayer), Pletaal tablet (Otsuka Pharm.), Disgren capsule (Myoung-In Pharmaceutical Co., Ltd.), Opalmon tablet (Dong A Pharmaceutical Co., Ltd.), etc., which are administered to over half of patients who were prescribed with antithrombotic agents in the years 2006-2007 in Korea.

Since the patent period for Plavix tablet had been expired, Aggrenox^{R} (Behringer Ingelheim) which is a complex drug comprising 200 mg dipyridamol and 25 mg asprin is becoming highlightened. Aggrenox^{R} is more potent than asprin and it was found to be remarkably effective for stroke adjustment patients.

However, there were no studies which isolate materials having antithrombitic activity from muskrat musk.

### DISCLOSURE OF INVENTION

### Technical Problem

With an effort to isolate a material having a potent antithrombotic activity from muskrat musk, the present inventors have found materials which have a higher antithrombotic activity than asprin.

It is an object of the present invention to provide a method of preparing an antithrombotic agent having a high potency from muskrat musk.

Another object of the present invention is to provide an antithrombotic composition comprising the antithrombotic agent.

### Technical Solution

In a first aspect, the present invention provides a method of preparing an antithrombotic agent from muskrat musk which comprises the steps of:
treating muskrat must with an organic solvent selected from ethanol, methanol, hexane and ethyl acetate for a shaking extraction and centrifuging the organic solvent extract to obtain a supernatant (1^{st} step);
carrying out the 1^{st} normal phase column chromatography for the supernatant to obtain fractions (2^{nd} step);
carrying out a thin layer chromatography for the fractions to determine material patterns of the fractions and divide them into groups (3^{rd} step);
measuring a thrombin time of the divided groups and selecting a group which show the highest antithrombotic activity (4^{th} step);
carrying out the 2^{nd} normal phase column chromatography for the selected group to obtain fractions (5^{th} step);
carrying out a thin layer chromatography for the fractions to determine material patterns of the fractions and divide them into groups (6^{th} step);
measuring a thrombin time of the divided groups and selecting a group which shows the highest antithrombotic activity (7^{th} step);
carrying out the 3^{rd} reverse phase column chromatography for the selected group to obtain fractions (8^{th} step);
carrying out a reverse phase thin layer chromatography for the fractions to determine material patterns of the fractions and divide them into groups (9^{th} step); and
measuring a thrombin time of the divided groups and selecting a group which shows the highest antithrombotic activity (10^{th} step).

In a second aspect, the present invention provides an antithrombotic agent having a high potency of a thrombin time over 40 seconds, which is obtained by the method described above.

In a third aspect, the present invention provides a composition for treating thrombosis which comprises the antithrombotic agent having a high potency according to the 2^{nd} embodiment.

Hereinafter, the present invention is further described in detail.

First, muskrat musk is cleanly collected and keeps it in a cold dark room.

The collected muskrat musk is treated with an organic solvent selected from ethanol, methanol, hexane and ethyl acetate for a shaking extraction and centrifuged to obtain a supernatant which is used as an organic solvent extract. The organic solvent is preferably used in an amount of 10 to 20 parts by weight based on 100 parts by weight of muskrat musk for the purpose of extraction effect.

The residue left after centrifugation is treated again with the same solvent for a shaking extraction and centrifuged to obtain a supernatant which is used as an organic solvent extract together with the supernatant prepared in the above step.

The shaking extraction is carried out, but not limited to, at 150 to 200 rpm for 20 to 24 hours which maximizes an effect of extraction. Particularly, a rotation of less than 150 rpm of rotation provides a weak shaking powder and a rotation of more than 200 rpm stains a machine, which are not preferred. The shaking extraction is preferably repeated three times or more.

The centrifugation is carried out, but not limited to, at 8,000 to 20,000 rpm for 10 to 20 minutes to maximize a precipitation of the residue, which is most preferred in the aspects of extraction effect and economics.

As for the 2^{nd} step, the organic solvent extract is carried out for the 1^{st} normal phase column chromatography to obtain column fractions. The column packing is a normal phase silicagel (230 to 400 mesh) and the organic solvent as a mobile phase includes, but not limited to, a solvent combination of hexane and another solvent which is more polar than hexane, for example, hexane and ethyl acetate, hexane and chloroform, hexane and acetone, and hexane and acetonitrile, ethyl acetate and ethanol, or ethyl acetate and methanol, wherein the combination ratio of said solvents is increased from 400:1 to 1:1 to obtain numerous fractions.

As for the 3^{rd} step, the fractions obtained in the 2^{nd} step is carried out for a thin layer chromatography to determine the material pattern of the fractions and divided into groups. The developing solvent for the thin layer chromatography is 10:1 to 10:2 of hexane:ether, or 3:1 to 3 of hexane:ethyl actetate, which is preferred in the aspect of developing effect (extraction effect).

The material patterning of the fractions is carried out through the following three steps in order:
a) exposing the fractions to a short wavelength (254 nm) lamp and a long wavelength (365 nm) UV lamp to identify a material pattern;
b) treating the plate used in the thin layer chromatography with 10% sulfuric acid and heating it to develop colored spots to identify the material pattern again; and
c) dividing the materials which have a similar moving distance on the basis of the patterns identified in step a) and step b) into groups.

As for the 4^{th} step, a thrombin time of the divided groups in the 3^{rd} step are measured and the group showing the highest antithrombotic activity is selected. The thrombin time is, but not limited to, a time until fibrinogen clots are formed. For example, with Thrombitimer2 (Behnk Elektronik, Germany), after adding Thrombin (T46481KU-59NIH unit/mg) and fibrinogen, the time until fibrinogen clots are formed is counted.

As for the 5^{th} step, the group selected from the 4^{th} step with the highest antithrombotic activity is carried out for the 2^{nd} normal phase column chromatography to obtain column fractions. The column packing is a normal phase silicagel (230 to 400 mesh) and the solvent as a mobile phase is, as shown above, selected from hexane and ethyl acetate, hexane and chloroform, hexane and acetone, and hexane and acetonitrile, ethyl acetate and ethanol, ethyl acetate and methanol, etc., wherein the combination ratio of said solvents is increased from 70:1 to 1:1, which is preferred in the aspect of extraction effect and economics.

As for the 6^{th} step, the fractions obtained in the 5^{th} step are carried out for a thin layer chromatography to determine the material patterns of the fractions and divided into groups. The developing solvent for the thin layer chromatography is, as shown above, 10:1 to 10:2 of hexane:ether, or 3:1 to 3 of hexane:ethyl actetate.

The material patterning of the fractions is carried out through the following three steps in order:
a) exposing the fractions to a short wavelength (254 nm) lamp and a long wavelength (365 nm) UV lamp to identify a material pattern;
b) treating the plate used in the thin layer chromatography with 10% sulfuric acid and heating it to develop colored spots to identify the material pattern again; and
c) dividing the materials which have a similar moving distance on the basis of the patterns identified in step a) and step b) into groups.

As for the 7^{th} step, a thrombin time of the divided groups in the 6^{th} step is measured and the group showing the highest antithrombotic activity is selected. The antitrombin time is, but not limited to, a time until fibrinogen clots are formed. For example, with Thrombitimer2 (Behnk Elektronik, Germany), after adding Thrombin (T46481KU-59NIH unit/mg) and fibrinogen, the time until fibrinogen clots are form is counted.

As for the 8^{th} step, the selected group is carried out for the 3^{rd} reverse phase column chromatography to obtain column fractions. The column packing is a reverse phase silicagel (63 to 212 [symbol] ) and the solvent as a mobile phase is an organic solvent selected from acetonitrile, methanol, ethanol and acetone and mixed with water, wherein the combination ratio of said organic solvent and water is increased from 20:1 to 1:1, which is preferred in the aspect of extraction effect and economics.

As for the 9^{th} step, the fractions obtained in the 8^{th} step are carried out for a reverse phase thin layer chromatography to determine the material pattern of the fractions and divided into groups. The developing solvent for the thin layer chromatography is 40:1 to 60:1 of acetonitrile:water, or 1:1 to 2:1 of ethyl acetate:methanol.

The material patterning of the fractions is carried out, as shown above, through the following three steps in order:
a) exposing the fractions to a short wavelength (254 nm) lamp and a long wavelength (365 nm) UV lamp to identify a material pattern;
b) treating the plate used in the thin layer chromatography with 10% sulfuric acid and heating it to develop colored spots to identify the material pattern again; and
c) dividing the materials which have a similar moving distance on the basis of the patterns identified in step a) and step b) into groups.

As for the 10^{th} step, a thrombin time of the divided groups in the 9^{th} step is measured and the group showing the highest antithrombotic activity is selected.

The antitrombin time is, but not limited to, a time until fibrinogen clot are formed. For example, with Thrombitimer2 (Behnk Elektronik, Germany), after adding Thrombin (T46481KU-59NIH unit/mg) and fibrinogen, the time until fibrinogen clots are formed is counted.

As shown in FIG. 8, the obtained antithrombotic agent has at least 40 seconds of thrombin time/0.002 mg of the agent and shows a remarkably superior antithrombotic effect over three times at a dilution of 50 times, compared with asprin which has 15 seconds of thrombin time/0.1 mg of asprin.

Although the exact structure of the agent have yet to be defined, it is assumed that the agent belongs to fatty acids having 25 carbon atoms, 48 hydrogens and 2 oxygens.

Furthermore, the present invention provides a composition for treating thrombosis which comprises the antithrombotic agent having a high potency. When the conventional components which are known as a drug having an antithrombotic effect are mixed with the composition, a synergistic effect in treating thrombosis is expected.

### Advantageous Effects

As discussed above, the present invention provides an antithrombotic agent having a high potency compared with asprin wherein the agent is obtained from muskrat musk and a composition for treating thrombosis which comprises the agent, and thus can raise value added of muskrat musk.

### BRIEF DESCRIPTION OF THE DRAWINGS

FIG. 1 is a flow chart showing the process for obtaining a solvent extract from muskrat musk.

FIG. 2 is a flow chart showing the process for separating an antithrombotic material from muskrat musk.

FIG. 3 shows the result of a thin layer chromatography of the fractions obtained from the 1^{st} normal phase column chromatography, wherein the result is grouped.

FIG. 4 is a graphic diagram showing the thrombin time of the fractions obtained from the 1^{st} normal phase column chromatography shown in FIG. 3.

FIG. 5 shows the result of a thin layer chromatography of the fractions obtained from the 2^{nd} normal phase column chromatography of the fraction B1-5 shown in FIG. 4, wherein the result is grouped.

FIG. 6 is a graphic diagram showing the thrombin time of the fractions obtained from the 2^{nd} normal phase column chromatography shown in FIG. 5.

FIG. 7 shows the result of a reverse phase thin layer chromatography of the fractions obtained from the 3rd reverse phase column chromatography of the fraction B2-6 shown in FIG. 6, wherein the result is grouped.

FIG. 8 is a graphic diagram showing the thrombin time of the fractions obtained from the 3^{rd} column chromatography shown in FIG. 7.

### BEST MODE FOR CARRYING OUT THE INVENTION

Material

The muskrat must used in the present invention was collected from the muskrats at Pusan University attached muskrat-run. The column packing used for a normal phase column was Merck (germany) silicagel (230 to 400 mesh, ASTM) and the column packing used for a reverse phase column was Wako (japan) RP-18 (63 to 212 [symbol] ) silicagel. To determine the antithrombotic activity of the fractions obtained from column chromatographies, Thrombitimer2 (Behnk Elektronik, Germany) was used in counting the thrombin time.

### Example 1

### Ethanol Extraction of Muskrat Musk

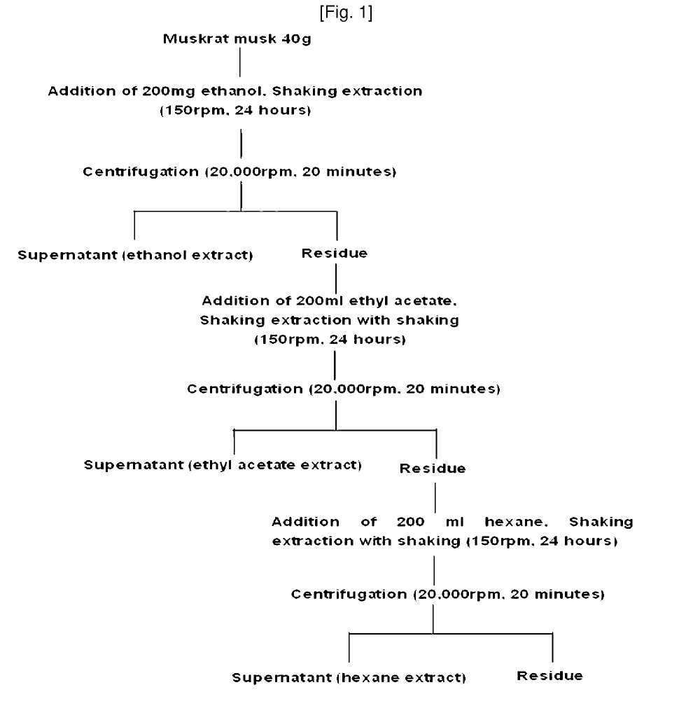

Referring to FIG. 1, sequential extractions with organic solvents were carried out for the muskrat musk which was clearly collected from muskrat and kept in a cold dark room.

Specifically, 40 g of muskrat musk was added into an Erlenmeyer flask, ethanol (5 times (200 ml) of the weight of muskrat musk) was added thereto, and the mixture was shaken at 150 rpm for 24 hours and centrifuged at 20,000 rpm to obtain a supernatant.

To repeat the ethanol extraction an equal amount of ethanol was added to the residue, so that another ethanol extract in addition to the above ethanol extract was obtained.

200 ml of ethyl acetate was added to the residue and, according to the method as shown in the ethanol extraction, an ethyl acetate extract was collected. Further, 200 ml of hexane was added to the residue and, according to the method as shown in ethanol extraction, a hexane extract was collected.

### Mode for the Invention

### Example 2

### 1^{st} Normal Phase Column Chromatograph and Thin Layer Chromatography of the Ethanol Extract from Muskrat Musk

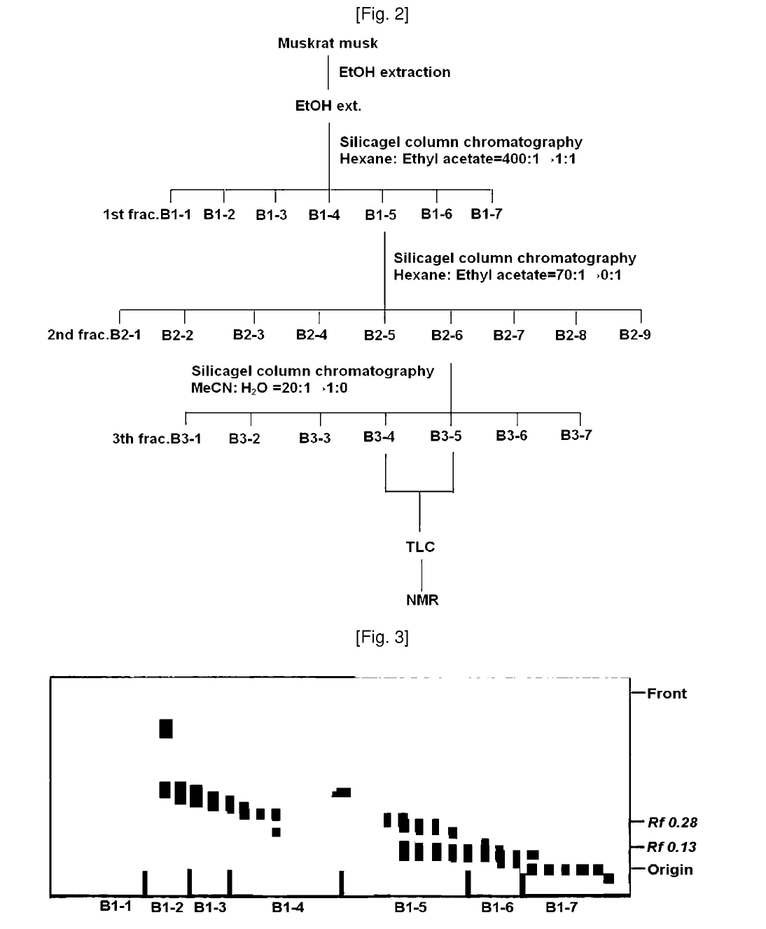

Referring to FIG. 2, the ethanol extract obtained in Example 1 was fractionized by the 1^{st} normal phase column chromatography.

The column packing was a normal phase silicagel (230 to 400 mesh) and the solvent as a mobile phase was hexane and ethyl acetate wherein the combination ratio of said solvents was increased from 400:1 to 1:1 to obtain 116 fractions.

To identify a material pattern of the fractions a thin layer chromatography was carried out and hexane and ether (10:1.5) was used as a developing solvent.

Further, the pattern of the fractions was identified by a short wavelength UV lamp (254 nm) and a long wavelength UV lamp (365 nm). The pattern was again identified by the spots colored after treating the thin layer chromatography plate with 10% sulfuric acid and heating it.

Based on the patterns identified above, the fractions were divided into 7 groups which have a similar migration distance, that is, groups B1-1 to 7, as shown in FIG. 3.

Antithrombotic activity for the 7 groups was studied.

The activity was measured by counting a thrombin time according to the method described in Choi et al., Kor. J. Pharmacogn. 26, 154-158, 1995 and Son et al., Kor. J. Pharmacogn. 34 (1):52-61, 2004.

Specifically, the thrombin time was measured with Thrombitimer2 (Behnk Elektronik, Germany) wherein a fixed concentration of specimen was dissolved in 10 [symbol] of dimethylsulfoxide (DMSO), the solution was added to a tube with 50 [symbol] of borate buffer (pH7.8), Thrombin 1 unit/50 [symbol] (T46481KU-59NIH unit/mg solid, Sigma Co., USA) was mixed with the solution, the mixture was warmed at 37° C. for 5 minutes, 200 [symbol] of fibrinogen (0.33%, Sigma Co., USA) was added to it, and the time until coaggulation occurs was counted.

Water and DMSO which is used to dissolve specimens were used as control groups and asprin (Sigma Co., USA) was used as a test control group compared with the fractions separated from the columns.

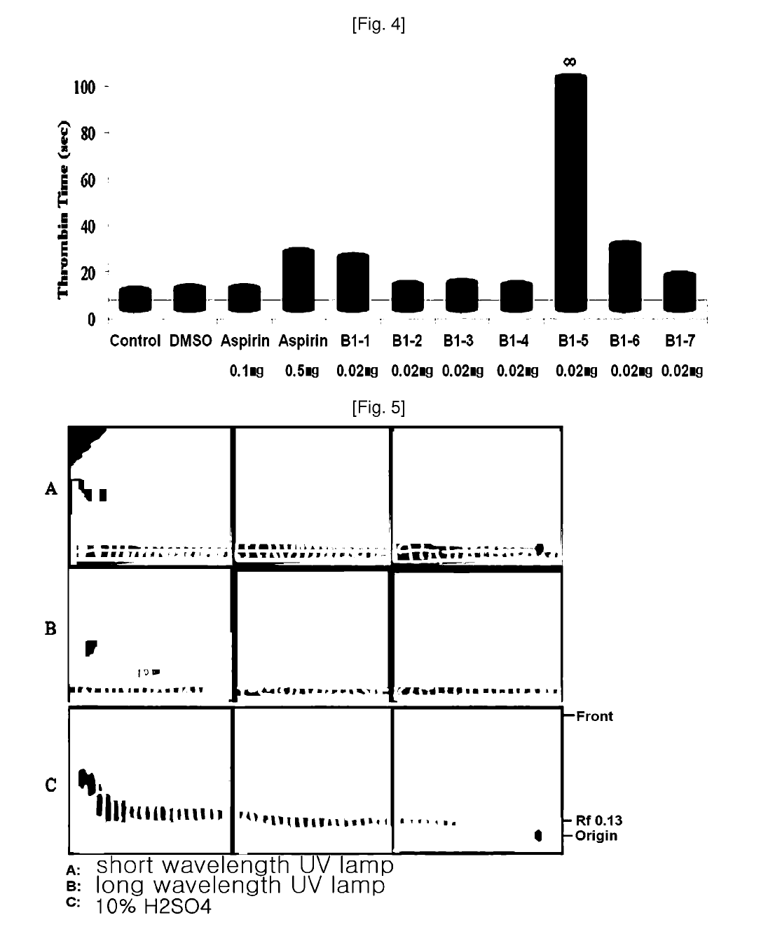

Antithrombotic activity was measured by counting a thrombin time and the result is an average of at least 3 experiments. The result is shown in FIG. 4.

The group B1-5 showed a higher antithrombotic activity than asprin.

### Example 3

### 2^{nd} Normal Phase Column Chromatography and Thin Layer Chromatography of the 1^{st} Fraction

Referring to FIG. 2, the group B1-5 in Example 2 which showed a higher antithrombotic activity than asprin was fractionized by the 2^{nd} normal phase column chromatography.

The solvent as a mobile phase was hexane and ethyl acetate wherein the combination ratio of said solvents was increased from 70:1 to 1:1 to obtain 172 fractions.

The pattern for the fractions obtained in the above was identified according to the method as shown in Example 2 and the result is given in FIG. 5. The fractions were divided into 9 groups (B2-1 to 9) according to the migration distance and used in the measurement of antithrombotic activity.

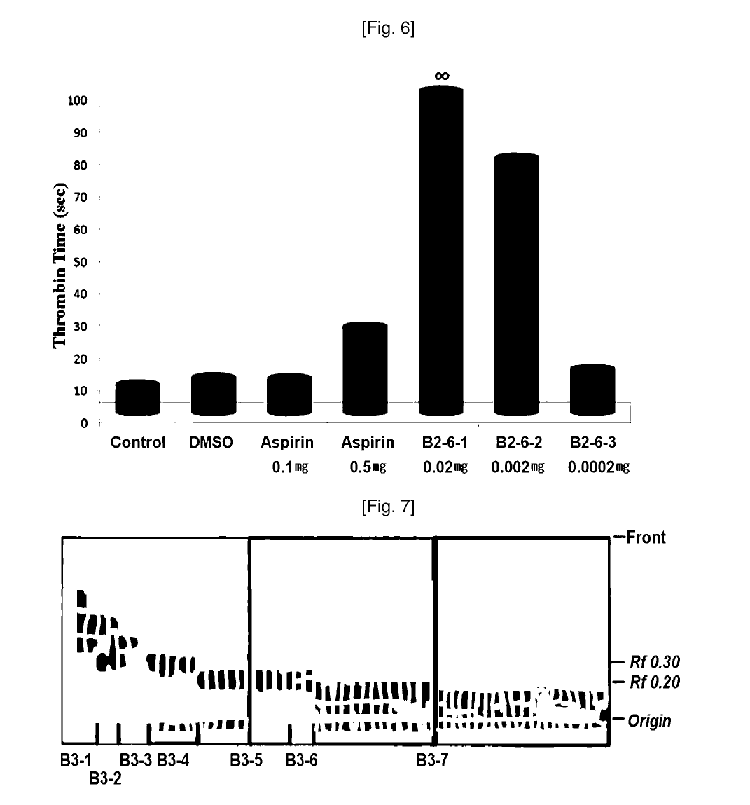

Antithrombotic activity for the material obtained from the 2^{nd} normal phase column was measured according to the method in Example 2 and the material in group B2-6 showed a higher antithrombotic activity over 2 times at a dilution of 250 times compared with asprin, as shown in FIG. 6.

### Example 4

### 3^{rd} Reverse Phase Column Chromatography and Thin Layer Chromatography of the 2^{nd} Fraction

Referring to FIG. 2, the group B2-6 in Example 3 which showed a higher antithrombotic activity than asprin was fractionized by a reverse phase column chromatography.

Reverse phase silicagel (63 to 212 [symbol] ) was used as a packing and the solvent as a mobile phase was acetonitrile and water wherein the combination ratio of said solvent was increased from 20:1 to 1:0 to obtain 234 fractions.

To identify a material pattern of the fractions a reverse phase thin layer chromatography was carried out and the pattern of the fractions was identified by a short wavelength UV lamp (254 nm) and a long wavelength UV lamp (365 nm). The pattern was again identified by the spots colored after treating the thin layer chromatography plate with 10% sulfuric acid and heating it.

Based on the patterns identified above, the fractions were divided into 7 groups which have a similar migration distance in the thin layer chromatography, that is, groups B3-1 to 7, as shown in FIG. 7.

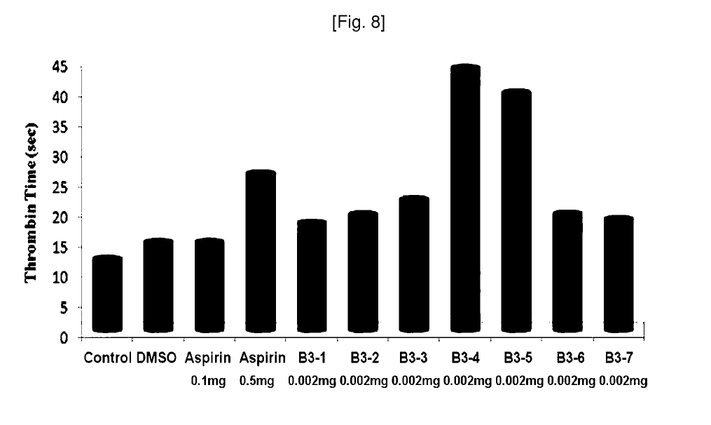

Antithrombotic activity was measured according to the method as shown in Example 2 and the groups B3-4 and B3-5 showed a higher antithrombotic activity than asprin, as shown in FIG. 8. Notably, the specimen in the groups B3-4 and B3-5 showed at least 40 seconds of thrombin time/0.002 mg of the specimen, which proves a remarkably superior antithrombotic effect over three times at a dilution of 50 times, compared with asprin which has 15 seconds of thrombin time/0.1 mg of asprin.

### INDUSTRIAL APPLICABILITY

The present invention provides an antithrombotic agent having a high potency compared with asprin, which is obtained from muskrat musk, and an antithrombotic composition comprising the agent, and thus can raise value added of muskrat musk.

1. A method of preparing an antithrombotic agent from muskrat musk which comprises the steps of
treating muskrat must with an organic solvent selected from ethanol, methanol, hexane and ethyl acetate for a shaking extraction and centrifuging the organic solvent extract to obtain a supernatant (1^{st} step);
carrying out the 1^{st} normal phase column chromatography for the supernatant to obtain fractions (2^{nd} step);
carrying out a thin layer chromatography for the fractions to determine material patterns of the fractions and divide them into groups (3^{rd} step);
measuring a thrombin time of the divided groups and selecting a group which show the highest antithrombotic activity (4^{th} step);
carrying out the 2^{nd} normal phase column chromatography for the selected group to obtain fractions (5^{th} step);
carrying out a thin layer chromatography for the fractions to determine material patterns of the fractions and divide them into groups (6^{th} step);
measuring a thrombin time of the divided groups and selecting a group which shows the highest antithrombotic activity (7^{th} step);
carrying out the 3^{rd} reverse phase column chromatography for the selected group to obtain fractions (8^{th} step);
carrying out a reverse phase thin layer chromatography for the fractions to determine material patterns of the fractions and divide them into groups (9^{th} step); and
measuring a thrombin time of the divided groups and selecting a group which shows the highest antithrombotic activity (10^{th} step).

2. The method of claim 1, wherein the organic solvent for the shaking extraction in the 1^{st} step is 10 to 20 parts by weight based on 100 parts by weight of muskrat musk.

3. The method of claim 1, wherein the shaking extraction in the 1^{st} step is carried out at 150 to 200 rpm for 20 to 24 hours.

4. The method of claim 1, wherein the centrifugation in the 1^{st} step is carried out at 8,000 to 20,000 rpm for 10 to 20 minutes.

5. The method of claim 1, wherein the residue left after centrifugation is treated again with the same solvent as that used in the previous extraction step for the shaking extraction and thereafter the extract is centrifuged to obtain a supernatant which is used in the 2^{nd} step together with the supernatant prepared in the previous extraction step.

6. The method of claim 1, wherein the 1^{st} normal phase column chromatography in the 2^{nd} step is carried out with a normal phase silicagel (230 to 400 mesh) and, as a mobile phase, an organic solvent combination selected from hexane and ethyl acetate, hexane and chloroform, hexane and acetone, and hexane and acetonitrile, ethyl acetate and ethanol, or ethyl acetate and methanol, wherein the combination ratio of said solvents is increased from 400:1 to 1:1.

7. The method of claim 1, wherein the developing solvent for the thin layer chromatography in the 3^{rd} and 6^{th} steps is 10:1 to 2 of hexane:ether or 3:1 to 2 of hexane:ethyl acetate.

8. The method of claim 1, wherein the developing solvent for the thin layer chromatography in the 9^{th} step is 40:1 to 60:1 of acetonitrile:water or 1:1 to 2:1 of ethyl acetate:methanol.

9. The method of claim 1, wherein the material patterning of the fractions in the 3^{rd}, 6^{th} and 9^{th} steps is carried out through the following three steps of
a) exposing the fractions to a short wavelength (254 nm) lamp and a long wavelength (365 nm) UV lamp to identify a material pattern;
b) treating the plate used in the thin layer chromatography with 10% sulfuric acid and heating it to develop colored spots to identify the material pattern again; and
c) dividing the materials which have a similar moving distance on the basis of the patterns identified in step a) and step b) into groups.

10. The method of claim 1, wherein the thrombin time in the 4^{th}, 7^{th} or 10^{th} steps is measured by counting the time until fibrinogen clots are formed after adding thrombin and fibrinogen of Thrombitimer2 product.

11. The method of claim 1, wherein the 2^{nd} normal phase column chromatography in the 5^{th} step is carried out with a normal phase silicagel (230 to 400 mesh) and, as a mobile phase, an organic solvent combination selected from hexane and ethyl acetate, hexane and chloroform, hexane and acetone, and hexane and acetonitrile, ethyl acetate and ethanol, or ethyl acetate and methanol, wherein the combination ratio of said solvents is increased from 70:1 to 1:1.

12. The method of claim 1, wherein the reverse phase column chromatography in the 8^{th} step is carried out with a reverse phase silicagel (63 to 212 [symbol] ) an, as a mobile phase, a solvent combination of an organic solvent selected from acetonitrile, methanol, ethanol and acetone and water, wherein the combination ratio of said solvents is increased from 20:1 to 1:1.

13. An antithrombotic agent which is prepared by the method according to any one of claims 1 to 12, wherein the agent has more than 40 seconds of thrombin time.

14. An composition for treating thrombosis which comprises the antithrombotic agent of claim 13.
